(19) United States
(12) Patent Application Publication
(10) Pub. No.: US 2003/0225282 A1
(43) Pub. Date: Dec. 4, 2003

(54) Enantioselective process for preparing arylated lactones and derivatives

(76) Inventors: Tony Yantao Zhang, Indianapolis, IN (US); Hongbin Zhang, Kunming (CN); Christophor Scott Proctor, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/220,444

(22) PCT Filed: Mar. 12, 2001
(86) PCT No.: PCT/US01/05800

(51) Int. Cl. C 07D 4 9/02; C07D 241/04; C07D 307/02

(52) U.S. Cl. 544/392; 549/295; 549/60

(57) ABSTRACT

This invention provides a process for the arylation of lactones to form to chiral and achiral aryllactones having high enantioselectivity where applicable.

### FIELD OF THE INVENTION

This invention relates to the art of synthetic organic chemistry. Specifically, the invention is a process to enantioselectively prepare arylated lactones of formula I:

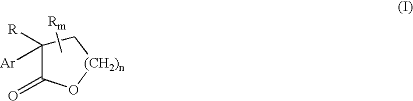

### BACKGROUND OF THE INVENTION

Lactones, substituted lactones and derivatives thereof, or compounds containing the lactone functionality are important medicinal agents or intermediates for the preparation of medicinal agents. For example, (−)-physostigmine, an alkaloid obtained from the Calabar bean fruit, has been found to be useful in the treatment of various clinical indications, including for example as a cholinesterase inhibitor.

Synthesis of chiral compounds with absolute stereocontrol remains a challenge. In particular, no high yield, highly enantioselective procedure has been reported for the synthesis of arylated lactones with absolute stereocontrol. Takano et. al., Che. Pharm. Bull. 30(7) 2641-2643, 1992 reported a synthesis of physostigmine by using a chiral synthon (s)-(−)-benzyl 2,3-epoxypropyl ether. In that process the epoxide, s-(−)-benzyl-2,3-epoxypropyl ether was ring opened with a carbanion prepared in-situ from 3-methoxybenzyl cyanide to afford an epimeric mixture of cyano alcohol, which was then lactonized by treatment with ethanolic base solution to provide the lactone backbone of physostigmine.

Buchwald et. al., J. Am. Chem. Soc. 120, 1918-1919, 1998, reported a catalytic assymetric arylation of ketone enolates using catalytic palladium(0) catalysts in the presence of aryl bromides. Burchwald et. al., Ibid. reported the enantioselective arylation of ketones, for example, 2-methyl-α-tetralone with 1-bromo-4-parabutylbenzene. The process required the use of 10-20% palladium (0)/12-24 mol % BINAP in toluene at 100° C., to afford a 73% yield of the desired arylated tetralone at 88% enantiomeric excess.

Hartwig et. al., J. Am. Chem. Soc. 119, 12382-12383, 1997, have reported the effect of sterically hindered chelating ligands in accelerating the rate of arylation of ketones using palladium catalysis.

Facile, enantioselective arylation of lactones continue to be a challenge and has not been reported. The ability to enantioselectively arylate lactones would be desirable for the synthesis of drug candidates and intermediates having an aryllactone moiety.

### SUMMARY OF THE INVENTION

The present invention provides a process for preparing a compound of the formula (I):

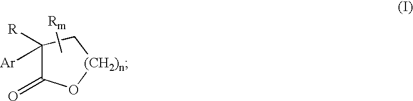

or pharmaceutically acceptable salts thereof, wherein R is a H, alkyl, alkenyl, alkynyl, aryl, alkylaryl, arylalkyl, heteroalkyl, heteroaryl, carboxyalkyl, carboxyaryl, cyano, carbocyclic or heterocyclic radical;

Ar is an aryl group;

Rₘ is a single or multiple substituent on the lactone ring other than at the α- position; and

n is 1-20, comprising the steps of:

(a) stirring a mixture of palladium source and a chiral ligand in a suitable solvent;

(b) adding a compound of formula ArX, wherein Ar is aryl, X is the anion of a strong acid or a leaving group selected from the group comprising Br, Cl, I, OSO₂ CₙF₂ₙ₊₁, and OP(O)(OCH₂ₙ₊₁)₂;

(c) adding a lactone;

(d) adding a suitable base

(e) isolating the product mixture from the reaction mixture;

(f) optionally preparing a pharmaceutically acceptable acid salt.

The present invention also provides a process for the preparation of a compound of formula (II)

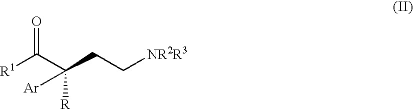

wherein R and R¹ are hydrogen, alkyl, alkenyl, alkynyl, aryl, alkylaryl, arylalkyl, heteroalkyl, heteroaryl, carboxyalkyl, carboxyaryl, cyano, carbocyclic or heterocyclic radical or combine to form a substituted or unsubstituted carbocycle or heterocycle;

R² and R³ are independently selected from the group consisting of H, alkyl, alkenyl, alkynyl, aryl, alkylaryl, arylalkyl, heteroalkyl, heteroaryl, carboxyalkyl, carboxyaryl, cyano, carbocyclic or heterocyclic radical or combine to form a substituted or unsubstituted carbocycle or heterocycle, comprising the steps of:

(a) arylating a compound of formula (a)

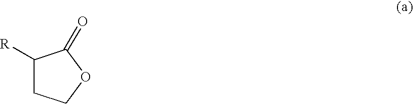

with an aryl halide (ArX) in the presence of a palladium source and a chiral ligand, in a suitable solvent to form a compound of formula (b)

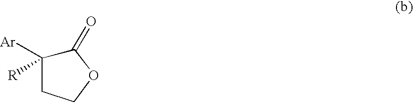

wherein R and Ar are as described;

(b) performing a 1,2-addition on the lactone to form the compound (c)

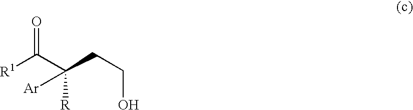

wherein R¹ is as described;

(c) oxidizing the compound (c) to afford the compound of formula (d)

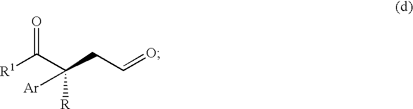

(d) performing a reductive amination using an amine (NHR²R³) and a reducing agent to form a compound of formula (II)

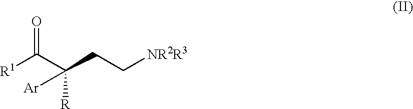

wherein R, R¹, R² and R³ are as described; and

(e) optionally forming a salt of a compound of formula (II).

The present invention is an enantioselective process for the preparation of α-arylated lactones

The present invention is an enantioselective process for the preparation of intermediates useful in the synthesis of pharmaceutically active compound.

### Definitions

The terms and abbreviations used herein have their normal meanings unless otherwise designated. For example “° C.” refers to degrees Celsius; “N” refers to normal or normality; “mmol” refers to millimole or millimoles; “g” refers to gram or grams; “d” refers to density, “min.” refers to minutes, “mL” means milliliter or milliliters; “M” refers to molar or molarity; “HPLC” refers to high performance liquid chromatography; “mm” refers to millimeters;

The term “halo” refers to fluoro, bromo, chloro and iodo.

As used herein BINAP is 2,2′-bis(diphenylphosphino)-1,1′-binaphtyl, used either as the S or R enantiomer as indicated.

As used herein, the terms “heterocycle” or “heterocyclic radical” refer to radicals derived from monocyclic or polycyclic, saturated or unsaturated, substituted or unsubstituted heterocyclic nuclei having 5 to 14 ring atoms and containing from 1 to 3 hetero atoms selected from the group consisting of nitrogen, oxygen or sulfur. Typical heterocyclic radicals are pyridyl, thienyl, fluorenyl, pyrrolyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, thianaphtheneyl, dibenzothiophenyl, indazolyl, imidazo(1.2-A)pyridinyl, benzotriazolyl, anthranilyl, 1,2-benzisoxazolyl, benzoxazolyl, benzothiazolyl, purinyl, pryidinyl, dipyridylyl, phenylpyridinyl, benzylpyridinyl, pyrimidinyl, phenylpyrimidinyl, pyrazinyl, 1,3,5-triazinyl, quinolinyl, phthalazinyl, quinazolinyl, and quinoxalinyl. The term “carbocyclic radical” refers to radicals derived from a saturated or unsaturated, substituted or unsubstituted 5 to 14-membered organic nucleus whose ring forming atoms (other than hydrogen) are solely carbon atoms. Typical carbocyclic radicals are cycloalkyl, cycloalkenyl, phenyl, naphthyl, norbornanyl, bicycloheptadienyl, tolulyl, xylenyl, indenyl, stilbenyl, terphenylyl, diphenylethylenyl, phenylcyclohexeyl, acenaphthylenyl, and anthracenyl, biphenyl, bibenzylyl and related bibenzylyl homologues represented by the formula (bb),

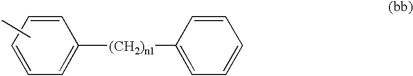

where n1 is an integer from 1 to 8.

As used herein the term “aryl” retains its commonly understood meaning of cyclic groups having the 4n+2 pi electronic structure and includes for example, substituted or unsubstituted aromatic radical selected from the group comprising 2-furyl, 3-furyl, 2-thienyl 3- thienyl, 1-pyrrolyl, 2-pyrrolyl, 3-pyrrolyl, phenyl, 2-pyridyl, 3-pyridyl, 4-pyridyl, 1-naphthyl, 2-naphthyl, 2-benzofuryl, 3-benzofuryl, 4-benzofuryl, 5-benzofuryl, 6-benzofuryl, 7-benzofuryl, 2-benzothienyl, 3-benzothienyl, 4-benzothienyl, 5-benzothienyl, 6-benzothienyl, 7-benzothienyl, 1-indolyl, 2-indolyl, 3-indolyl, 4-indolyl, 5-indolyl, 6-indolyl, 7-indolyl, and 8-indolyl. The optional substitutions of aryl may be at one or two carbon atoms of the aryl group, and may be with heterocyclic radical, C₁₋₄ alkyl, C₁₋₄ alkoxy, halogen, —NO₂, —CN, —COOH, —CONH₂, —SR⁹, —OR¹⁰, —SO₃H, —SO₂NH₂ or trifluoromethyl, R⁹ and R¹⁰ are independently hydrogen, —CF₃, phenyl, —(C₁-C₄)alkyl, —(C₁-C₄)alkylphenyl or -phenyl(C₁-C₄)alkyl. Examples of substituted aryl groups are 4-methyl-3-furyl, 3,4-dimethyl-2-thienyl, 2,4-dimethyl-3-thienyl, 3-ethoxy-4-methyl-2-benzofuryl, 2-cyano-3-benzofuryl, 4-trifluoromethyl-2-benzothienyl, 2-chloro-3-benzothienyl, 3,4-dichloro-2-pyridyl, 2-bromo-3-pyridyl, 2-fluoro-4-pyridyl, 4-fluoro-2-furyl, 2-carboxyphenyl, 4-carboxamidophenyl, 3-trifluoromethylphenyl, bromo-1-naphthyl, 2,3-dimethyl-1-naphthyl, 3-carboxy-2-naphthyl, 5-carboxy-8-chloro-1-naphthyl, 3-ethyl-2-furyl, 8-fluoro-2-naphthyl, 5-trifluoromethyl-2-naphthyl, 6-ethoxy-2-naphthyl, 6,7-dimethoxy-2-naphthyl, 3-carboxy-2-naphthyl, and the like.

As used herein, the term “non-interfering sustituent” refers to radicals suitable for substitution on the lactone nucleus (depicted in Formula I) and radical(s) suitable for substitution on the heterocyclic radical and carbocyclic radical as defined above. Illustrative non-interfering radicals are hydrogen, —(C₁-C₁₄)alkyl, —(C₂-C₆)alkenyl, —(C₂-C₆)alkynyl, —(C₇-C₁₂)aralkyl, —(C₇-C₁₂)alkaryl, —(C₃-C₈)cycloalkyl, —(C₃-C₈)cycloalkenyl, phenyl, tolulyl, xylenyl, biphenyl, —(C₁-C₆)alkoxy, —(C₂-C₆)alkenyloxy, —(C₂-C₆)alkynyloxy, —(C₁-C₁₂)alkoxyalkyl, —(C₁-C₁₂)alkoxyalkyloxy, —(C₁-C₁₂)alkylcarbonyl, —(C₁-C₁₂)alkylcarbonylamino, —(C₁-C₁₂)alkoxyamino, —(C₁-C₁₂)alkoxyaminocarbonyl, —(C₁-C₁₂)alkylamino, —(C₁-C₆)alkylthio, —(C₁-C₁₂)alkylthiocarbonyl, —(C₁-C₆)alkylsulfinyl, —(C₁-C₆)alkylsulfonyl, —(C₁-C₆)haloalkoxy, —(C₁-C₆)haloalkylsulfonyl, —(C₁-C₆)haloalkyl, —(C₁-C₆)hydroxyalkyl, —(CH₂)ₙCN, —(CH₂)ₙNR⁹R¹⁰, —C(O)O(C₁-C₆alkyl), —(CH₂)ₙO(C₁-C₆alkyl), benzyloxy, phenoxy, phenylthio; —(CONHSO₂)R¹⁵, where R¹⁵ is —(C₁-C₆)alkyl; —CF₃, naphthyl or —(CH₂),phenyl where s is 0-5; —CHO, —CF₃, —OCF₃, pyridyl, amino, amidino, halo, carbamyl, carboxyl, carbalkoxy, —(CH₂)ₙCO₂H, cyano, cyanoguanidinyl, guanidino, hydrazide, hydrazino, hydrazido, hydroxy, hydroxyamino, nitro, phosphono, —SO₃H, thioacetal, thiocarbonyl, furyl, thiophenyl —COR⁹, —CONR⁹R¹⁰, —NR⁹R¹⁰, —NCHCOR⁹, —SO₂R⁹, —OR⁹, —SR⁹, CH₂SO₂R⁹, tetrazolyl or tetrazolyl substituted with —(C₁-C₆)alkyl, phenyl or —(C₁-C₄)alkylphenyl, —(CH₂)ₙOSi(C₁-C₆)alkyl and (C₁-C₆)alkylcarbonyl; where n is from 1 to 8 and R⁹ and R¹⁰ are independently hydrogen, —CF₃, phenyl, —(C₁-C₄)alkyl, —(C₁-C₄)alkylphenyl or -phenyl(C₁-C₄)alkyl

The term, “heterocyclic”, refers to radicals derived from monocyclic or polycyclic, saturated or unsaturated, substituted or unsubstituted heterocyclic nuclei having 5 to 14 ring atoms and containing from 1 to 3 hetero atoms selected from the group consisting of nitrogen, oxygen or sulfur. Typical heterocyclic radicals are pyrrolyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, thianaphtheneyl, dibenzothiophenyl, indazolyl, imidazo(1.2-A)pyridinyl, benzotriazolyl, anthranilyl, 1,2-benzisoxazolyl, benzoxazolyl, benzothiazolyl, purinyl, pryidinyl, dipyridylyl. phenylpyridinyl, benzylpyridinyl, pyrimidinyl, phenylpyrimidinyl, pyrazinyl, 1,3,5-triazinyl, quinolinyl, phthalazinyl, quinazolinyl, and quinoxalinyl.

As used herein, the term “pharmaceutically acceptable salt” refers to all non-toxic organic or inorganic acid addition salts. Illustrative inorganic acids or “acidic groups” which form salts include hydrochloric, hydrobromic, sulfuric, phosphoric acid and acid metal salts such as sodium monohydrogen orthophosphate, and potassium hydrogen sulfate. Illustrative acids or “acidic groups” which form suitable salts include the mono-, di- and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxy-benzoic, and sulfonic acids such as p-toluenesulfonic acid, methanesulfonic acid, camphorsulfonic acid, and 2-hydroxyethane sulfonic acid. Preferred acids include those selected from the group comprising of hydrobromic acid, hydrochloric acid, camphorsulfonic acid, p-toluenesulfonic acid, and sulfuric acid. A particularly preferred acidic group is hydrochloric acid. Acid addition salts formed from these acids can exist in either hydrated or substantially anhydrous form, all of which are within the scope of this invention.

As used herein, the term chiral ligand is synonymous with chiral auxillaries and refer to chiral compounds capable of forming chiral complexes with reactive agents e.g., palladium, to form chiral catalysts which aid in the stereo-differentiation of reactive sites and results in enantiomerically enriched reaction products. Chiral ligands or auxilliaries have been reviewed in the literature and are known to one of skill in the art. Examples of chiral ligands include but are not limited to chiral phosphines, chiral oxazolines, and chiral binaphthyl.

One of skill in the art is aware that while a particular enantiomer of a chiral ligand is exemplified or claimed, the opposite enantiomer may be used according to the process of the invention to afford the opposite enantiomer of product, or a preponderance thereof.

### DETAILED DESCRIPTION OF THE INVENTION

The present invention provides a process for preparing α-aryllactones via an enantioselective palladium catalyzed carbon-carbon bond formation between an aryl source and a lactone substrate.

The present invention provides a process for the preparation of compounds of formula 1

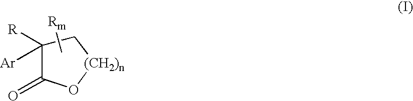

or pharmaceutically acceptable salts thereof,

wherein R is a hydrogen, alkyl, alkenyl, alkynyl, aryl, alkylaryl, arylalkyl, heteroaryl, carboxyalkyl, carboxyaryl, cyano, carbocyclic, heterocyclic radical;

Ar is an aryl group;

Rₘ is a single or multiple non-interfering substituent on the lactone ring other than at the α-position; and n is 1-20.

Preferred R groups for the purpose of the invention are the R groups selected from the group consisting of hydrogen, (C₁-C₈)alkyl, (C₁-C₁₄)alkylaryl, and arylalkyl groups.

Preferred Rₘ for the purpose of the present invention is a single or multiple non-interfering substituent independently selected from the group consisting of hydrogen, alkyl, alkenyl, alkynyl, aryl, alkylaryl, arylalkyl, alkylheterocyclic and arylheterocyclic radical. Most preferred Rₘ is a single or multiple non-interfering substituent independently (for multiple) selected from the group consisting of hydrogen, (C₁-C₈) alkyl, aryl, (C₁-C₁₄)alkylaryl.

Preferred for the purpose of the present invention is n=1, 2, or 3.

A preferred R¹ group for the purpose of the present invention is a non-interfering substituent selected from the group comprising of alkyl, alkenyl, alkynyl, aryl, heteroalky, alkylaryl, arylalkyl, alkylheterocyclic and arylheterocyclic radical. Most preferred R¹ is a non-interfering substituent selected from the group consisting of (C₁-C₈)alkyl, aryl, (C₁-C₁₄)alkylaryl.

Preferred aryl substrate or source(ArX) for the purpose of the present invention is the aryl substrate wherein X is a halogen, triflate or phosphonate. Most preferred is ArX wherein X is a bromide (Br), Iodide (I) or triflate (OSO₂CₙF₂ₙ₊₁).

Preferred R² and R³ groups are independently selected from the group consisting of H, alkyl, alkenyl, alkynyl, aryl, alkylaryl, arylalkyl, heteroalkyl, heteroaryl, carboxyalkyl, carboxyaryl, cyano, carbocyclic, heterocyclic or combine to with the nitrogen to which they are attached to form substituted or unsubstituted piperazinyl, piperidinyl, pyrrolidinyl, morpholino, or 1-hexamethyleneimino. Most preferred R² and R³ groups are independently selected from the group consisting of H, alkyl, alkenyl, alkynyl, aryl, alkylaryl, arylalkyl, heteroalkyl, heteroaryl or combine to with the nitrogen to which they are attached to form 4-(2-methoxyphenyl)piperazinyl, piperidinyl, pyrrolidinyl, methylpyrrolidinyl, dimethylpyrrolidinyl, morpholino, or 1-hexamethyleneimino.

Preferred palladium sources include elemental palladium, elemental palladium on supports including activated carbon, or alumina. Also preferred as palladium sources are palladium salts including but not limited to palladium acetate, palladium chloride, palladium bromide, and palladium complexes including but not limited to palladium bis(dibenzylideneacetone) palladium (0) (Pd(dba)₂), tris(dibenzylideneacetone) bispalladium (Pd₂(dba)₃), tetrakistriphenylphosphine palladium (0)(Pd(PPh₃)4), bistriphenylphosphinepalladium (II) chloride (Pd(Ph₃)₂Cl₂), and palladium bistriphenylphosphine diacetate (Pd(Ph₃)₂(OAc)₂).

Preferred chiral ligands are the chiral phosphines. A most preferred chiral ligand is BINAP, either the R or S depending on the desired configuration of reaction product.

A most preferred palladium catalyst system for the purpose of the present invention is 5-10% palladium acetate and 5-15% R or S BINAP depending on desired product configuration.

### Practice of the Invention

The practice of the invention involves the reaction of the enolate of a lactone with an aryl source in the presence of a palladium catalyst system in a suitable solvent. The lactone enolate may be generated in situ by reacting the lactone with a suitable base, preferably a strong base, more preferably an organic base such as for example, potassium bis(trimethylsilyl)amide (KN(TMS)₂) in a suitable solvent such as for example tetrahydrofuran or toluene or dioxane. This phase of the reaction is performed at a temperature of from about −80 to 150° C., preferably −20 to 30° C., and most preferably at about 20° C., depending on factors as solvent and base employed. Alternatively, the lactone enolate may be generated separately and cannulated to the reaction flask. The lactone enolate is then reacted with an aryl source such as for example aryl bromide, aryl iodide, aryltrifluoromethane sulfonate (aryl triflate) or aryl phosphonate in the presence of a palladium catalyst source, at about 80-120° C., over a period of 2 to 48 hours. Preferred aryl source include the aryl bromides, aryl iodides and aryl triflates. Preferred palladium catalyst source includes for example those generated from palladium acetate and a chiral ligand, and from Pd(dba)₂ and a chiral ligand. A preferred chiral ligand is R-(+)-BINAP or S-(−)-BINAP depending on the desired product configuration, and a preferred palladium source is palladium acetate. One of skill in the art is aware that an achiral ligand, a mixture of S and R ligands or lack of a ligand may result in a mixture of enatiomers and/or a low yield of aryl lactone. Thus, the present invention includes a novel process for the production of chiral and achiral aryllactones.

Typically, palladium acetate and R-(+)-BINAP were added to toluene and stirred at room temperature for about 20 to 80 minutes, preferably for about 60 minutes, and preferably under nitrogen. Aryl bromide and lactone (e.g., α-methyl-γ-butyrolactone) were added, preferably via syringe. A solution of a suitable base, preferably an organic base, preferably potassium bis(trimethylsilyl)amide was added drop-wise via syringe. The resulting mixture was heated at 40 to 120° C., preferably at about 100-105° C. for about 10 to 30 hours, preferably about 24 hours depending on the boiling point of the solvent chosen. The most preferred solvent is toluene. The reaction mixture was cooled, quenched with aqueous acid, preferably aqueous HCl and extracted. The product was chromatographed preferably on silica gel using a heptane/ethylacetate gradient. Alternatively, the reaction product could be isolated and purified by common laboratory techniques know to one of skill in the art.

The practice of the present invention to prepare a compound of formula (II),

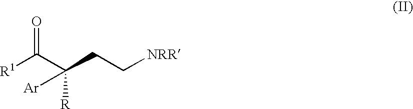

has been exemplified in a process to manufacture the compound of formula (X′) as shown in Scheme 1 below;

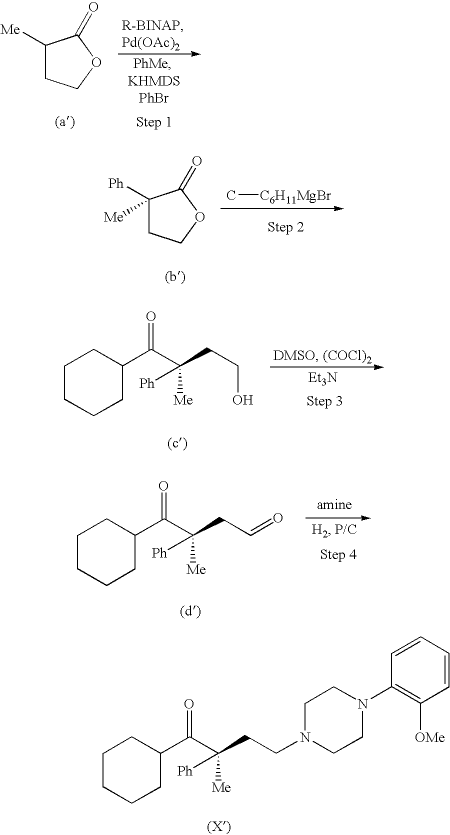

As shown in Scheme 1,2-methylbutyrolactone compound (a′) was arylated with phenyl halide, preferably phenyl bromide to form compound (b′). The process involved reacting the anion of 2-methylbutyrolactone (a′) generated by reacting the lactone with an organic base such as potassium bis(trimethylsilyl)amide, with R-BINAP and palladium acetate or other suitable palladium source. The resulting arylated lactone (b′) was subjected to a 1,2-addition reaction, i.e., reacted with cyclohexylmagnesium bromide to afford the ketone compound (c′). The Grignard reagent cyclohexylmagnesium bromide, was generated using cylcohexylbromide and magnesium tunings in the presence of an aprotic solvent preferably an ether (i.e., diethyl ether). Alternatives to Grignard reagents for the alkylation of lactones and/or procedures for generating Grignard reagents are known to one of skill in the art.

The alcohol (OH) group of the compound (c′) was oxidized using oxalylchloride, dimethylsufoxide and a tertiary amine base such as triethylamine (Swern oxidation)in a suitable solvant such as dichloromethane, typically at room temperature, to afford the aldehyde (d′). Several reagents and procedures exist for the oxidation of primary alcohols to aldehydes, and are known by one of skill in the art. For a review of alcohol oxidations see for example, Synthesis, 70, 1971, and Synthesis, 857, 1990).

The aldehyde (d′) was reductively aminated by reaction with the amine, 4-(2-methoxyphenyl) piperazine, under hydrogenation conditions to afford desired product of formula (X′). The reductive amination can be performed using suitable amines, reducing agents, and reaction conditions known to one of skill in the art. Reductive aminations may be performed step wise beginning with formation of the intermediate (often isolable) imine or enamine, and ending with reduction of the imine or enamine to the amine. Under certain conditions of substrate and reagents the two steps may be performed in the same reaction step.

Alcohol oxidations, Grignard reactions, reductive amination reactions are generally facile reactions, occurring at moderate temperatures and generally polar aprotic solvents. General references for these reactions include Advanced Organic Chemistry, 3^{rd} edition, by Jerry March, Wiley-Interscience Publishers, New York, N.Y., and Advanced Organic Chemistry, 3^{rd} edition, parts A and B, by Francis A. Carey and Richard J. Sundberg, Plenum Press, New York, N.Y.

To prepare the intermediate compound of formula III,

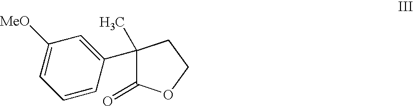

for example, the lactone compound of formula (a′)

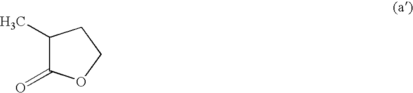

is reacted with about a molar equivalent of 3-bromoanisole (both available from Aldrich Chemical Company, Milwaukee, USA) in the presence of palladium acetate and R-BINAP (available from Aldrich Chemical Company, Milwaukee, USA). The mixture was heated in refluxing toluene over a period of 24 hours or as provided in the typical procedure below. The reaction scheme is shown below in scheme 2.

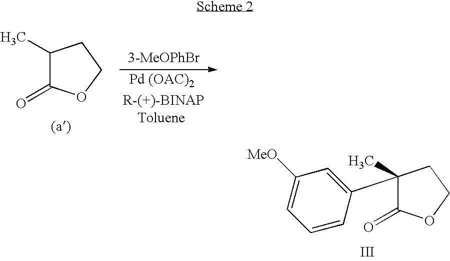

The product of formula III is a key intermediate in the synthesis of physostigmine. The intermediate III is converted to physostigmine by processes and procedures known to one of skill in the art and as described in Takano et. al., Che. Pharm. Bull. 30(7) 2641-2643, 1992.

One of skill in the art is aware that the order of performance of some steps of the process of the present invention are not critical and may be interchanged.

### EXAMPLES

The following examples and preparations are illustrative only and are not intended to limit the scope of the invention.

Typical Procedure: Palladium acetate (45 mg, 0.2 mmol, 0.1 eq.) and R-(+)-BINAP (156 mg, 0.25 mmol, 0.125 eq.) in dry toluene (30 mL, degased with dry nitrogen) were stirred at room temperature under nitrogen for 60 minutes. Aryl bromide (4 mmol, 2.0 eq.) and α-methyl-γ-butyrolactone (2 mmol) were added via syringe. KN(TMS)₂ in toluene (0.5 M, 7 mL, 3.5 mmol, 1.75 eq.) was added dropwise and the resultant dark red solution was then stirred at 100-105° C. for 24 hours. The reaction mixture was cooled to room temperature before treating with 1N HCl (15 mL) and water (50 mL). The mixture was extracted with ethyl acetate (3×50 mL) and the combined organic phase was washed with water (25 mL) and brine (40 mL) and dried over MgSO₄. After removal of the solvent, the residue was chromatographed on silica gel (heptane: ethyl acetate=8:1→2:1) to afford the product.

α-(3,4-Dimethoxyphenyl)-α-methyl-γ-butyrolactone: ¹H-NMR (300 MHz, CDCl₃) δ6.95 (1H, d, J=2.1 Hz); 6.90 (1H, dd, J=2.1, 8.4 Hz); 6.82 (1H, d, J=8.7 Hz); 4.32 (1H, ddd, J=3.9, 7.5, 9.0 Hz); 4.14 (1H, ddd, J=6.3, 8.7, 9.0 Hz); 3.88 (3H, s, OCH₃); 3.86 (3H, s, OCH₃); 2.66 (1H, ddd, J=3.9, 6.3, 12.6 Hz); 2.38 (1H, ddd, J=7.6, 8.5, 12.7 Hz); 1.58 (3H, s, CH₃). ¹³C-NMR (75 MHz, CDCl₃) δ179.96; 148.99; 148.18; 133.24; 117.79; 111.04; 109.40; 65.11; 56.00; 55.95; 47.11; 38.02; 25.89.

α-(3-Methoxyphenyl)-α-methyl-γ-butyrolactone : ¹H-NMR (300 MHz, CDCl₃) δ7.27 (1H, dd, J=8.1, 8.4 Hz); 6.94-6.99 (2H, m); 6.81 (1H, ddd, J=1.5, 2.4, 8.1 Hz); 4.31 (1H, ddd, J=3.9, 7.4, 8.7 Hz); 4.13 (1H, ddd, J=6.3, 8.4, 8.7 Hz); 3.80 (3H, s, OCH₃); 2.66 (1H, ddd, J=3.9, 6.3, 12.6 Hz); 2.38 (1H, ddd, J=7.6, 8.4, 12.6 Hz); 1.59 (3H, S, CH₃). ¹³C-NMR (75 MHz, CDCl₃) δ179.63; 159.63; 142.41; 129.65; 117.95; 112.24; 112.06; 64.99; 55.21; 47.44; 38.00; 25.46.

α-(4-Methylphenyl)-α-methyl-γ-butyrolactone: ¹H-NMR (300 MHz, CDCl₃) δ7.29 (2H, d, J=8.4 Hz); 7.17 (2H, d, J=8.4 Hz); 4.31 (1H, ddd, J=3.9, 7.5, 9.0 Hz); 4.12 (1H, ddd, J=6.3, 9.0, 9.0 Hz); 2.66 (1H, ddd, J=3.9, 6.4, 12.6 Hz); 2.38 (1H, ddd, J=7.6, 9.0, 12.6 Hz); 2.33 (3H, s, CH₃); 1.59 (3H, s, CH₃). ¹³C-NMR (75 MHz, CDCl₃) δ179.93; 137.78; 136.92; 129.34; 125.57; 64.98; 47.14; 38.03; 25.52; 20.96.

α-Methyl-α-(1-naphathyl)-γ-butyrolactone: ¹H-NMR (300 MHz, CDCl₃) δ7.95 (1H, d, J=8.1 Hz); 7.91 (1H, dd, J=2.1, 8.1 Hz); 7.81 (1H, d, J=8.4 Hz); 7.44-7.57 (3H, m); 7.41 (1H, dd, J=7.8, 7.9 Hz); 4.45 (1H, ddd, J=5.1, 7.8, 9.0 Hz); 4.29 (1H, ddd, J=7.2, 7.5, 9.0 Hz); 3.13 (1H, ddd, J=5.1, 7.2, 12.9 Hz); 2.47 (1H, ddd, J=7.5, 7.8, 12.9 Hz); 1.93 (3H, s, CH₃). ¹³C-NMR (75 MHz, CDCl₃) δ180.67; 135.78; 134.69; 129.57; 128.71; 125.71; 125.10; 124.71; 124.70; 124.05; 65.35; 47.77; 37.81; 24.08.

α-[2-(6-Methoxynaphathyl)]-α-methyl-γ-butyrolactone: ¹H-NMR (300 MHz, CDCl₃) δ7.75 (1H, d, J=8.7 Hz); 7.74 (1H, d, J=2.4 Hz); 7.71 (1H, d, J=9.0 Hz); 7.48 (1H, dd, J=2.4, 8.7 Hz); 7.16 (1H, dd, J=2.4, 9.0 Hz); 7.11 (1H, d, J=2.4 Hz); 4.34 (1H, ddd, J=3.6, 7.8, 9.0 Hz); 4.15 (1H, ddd, J=6.3, 8.9, 9.0 Hz); 3. 91 (3H, s, OCH₃); 2.75 (1H, ddd, J=3.6, 6.3, 12.9 Hz) ; 2.44 (1H, ddd, J=7.8, 9.0, 12.9 Hz); 1.68 (3H, s, CH₃). ¹³C-NMR (75 MHz, CDCl₃) δ179.89; 157.70; 135.60; 133.39; 129.33; 129.32; 128.34; 127.51; 124.31; 124.17; 119.09; 119.08; 105.28; 65.06; 55.26; 47.54; 38.01; 25.41.

α-Methyl-α-(2-naphathyl)-γ-butyrolactone: ¹H-NMR (300 MHz, CDCl₃) δ7.86 (1H, d, J=8.7 Hz); 7.83 (3H, m); 7.46-7.57 (3H, m); 4.35 (1H, ddd, J=3.9, 7.8, 9.0 Hz); 4.16 (1H, ddd, J=6.6, 9.0, 9.0 Hz); 2.77 (1H, ddd, J=3.9, 6.6, 12.6 Hz); 2.45 (1H, ddd, J=7.8, 8.7, 12.6 Hz); 1.70 (3H, s, CH₃). ¹³C-NMR (75 MHz, CDCl₃) δ179.74; 137.98; 132.91; 132.23; 128.69; 127.88; 127.30; 126.30; 126.11; 124.40; 123.81; 65.04; 47.73; 38.01; 25.36.

α-Biphenyl-α-methyl-γ-butyrolactone ¹H-NMR (300 MHz, CDCl₃) δ7.32-7.62 (9H, m); 4.36 (1H, ddd, J=3.9, 7.8, 9.0 Hz); 4.20 (1H, ddd, J=6.3, 8.4, 9.0 Hz); 2.73 (1H, ddd, J=3.9, 6.3, 12.6 Hz); 2.44 (1H, ddd, J=8.1, 8.4, 12.7 Hz); 1.66 (3H, s, CH₃). ¹³C-NMR (75 MHz, CDCl₃) δ179.78; 140.23; 139.86; 128.69; 127.40; 127.34; 126.92; 126.23; 65.07; 47.30; 38.05; 25.52.

α-(4-Cyanophenyl)-α-methyl-γ-butyrolactone: ¹H-NMR (300 MHz, CDCl₃) δ7.65 (2H, d, J=8.4 Hz); 7.56 (2H, d, J=8.4 Hz); 4.36 (1H, ddd, J=5.4, 7.8, 9.0 Hz); 4.18 (1H, ddd, J=7.2, 7.2, 9.0 Hz); 2.66 (1H, ddd, J=5.4, 7.2, 12.9 Hz); 2.45 (1H, ddd, J=7.2, 7.2, 12.9 Hz); 1.61 (3H, s, CH₃). ¹³C-NMR (75 MHz, CDCl₃) δ178.58; 146.34; 132.38; 132.37; 126.76; 126.75; 118.24; 111.23; 64.91; 47.42; 37.41; 25.19.

4-Cyclohexyl-3-methyl-4-oxo-3-phenyl-1-butanol

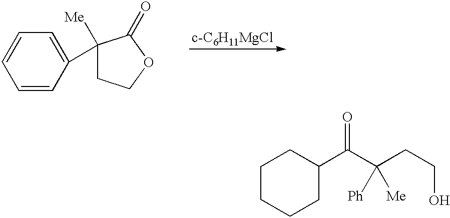

To a solution of 0.10 g (0.57 mmol) of 2-methyl-2-phenylbutyrolactone in 0.57 mL of toluene at reflux was added 0.28 mL (0.57 mmol) of 2M cyclohexylmagnesium chloride/Et₂O. After 47 min. the reaction mixture was cooled to RT, diluted with CH₂Cl₂, washed with 25 mL of 1N HCl (aq), washed with 25 mL of 25% NaCl (aq), dried over MgSO₄, gravity filtered and concentrated to afford 0.1403 g (94%) of product. NMR (d⁶-DMSO): δ7.17-7.39 (m, 5H, phenyl CH), 4.2-4.5 (br s, 1H, —OH), 3.20-3.25 (m, 1H, CH₂OH), 3.09-3.14 (m, 1H, CH₂OH), 2.34-2.38 (m, 1H, cyclohexyl CH), 1.97-2.05 (m, 2H, —CH₂CH₂OH), 1.43 (s, 3H, —CH₃), 0.84-1.54 (m, 10H, cyclohexyl CH).

4-Cyclohexyl-3-methyl-4-oxo-3-phenylbutyraldehyde

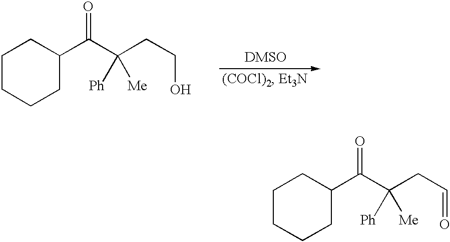

DMSO (0.92 mL, 0.013 mol) was added dropwise to a solution of 0.57 mL (0.0065 mol) of (COCl)₂ in 11 mL of CH₂Cl₂ (cooled below −60° C. in a dry ice acetone bath) over a 7 min period. After stirring below −60° C. for 23 min, a solution of 1.20 g (0.00463 mol) of 4-cyclohexyl-3-methyl-4-oxo-3-phenyl-1-butanol in 11 mL of CH₂Cl₂ and washings with 11 mL of CH₂Cl₂ was added dropwise to the reaction mixture below −60° C. over a 23 min period. After stirring below −60° C. for 24 min, 1.8 mL (0.013 mol) of Et₃N was added dropwise for 3 min to the yellow suspension. Cooling bath was removed and mixture became homogeneous until precipitate formed. After stirring for 3 h, the reaction mixture was partitioned between 50 mL of MTBE and 50 mL of 1N HCl (aq). The organic phase was washed with 50 mL of 1 N HCl (aq), washed with 50 mL of 25% NaCl (aq), dried over Mg SO₄, gravity filtered and concentrated to afford 1.18 g (98.3%) of product. ¹H NMR (d⁶-DMSO): δ9.50 (t, J=2.0 Hz, 1H, —CHO), 7.36-7.39 (m, 2H, phenyl CH), 7.27-7.30 (m, 3H, phenyl CH), 2.92 (dd, J=16.6 Hz, J=1.9 Hz, 1H, CH₂CHO), 2.82 (dd, J=16.6 Hz, J=1.7 Hz, 1H, CH₂CHO), 2.39-2.44 (m, 1H, cyclohexyl CH), 1.68 (s, 3H, R₂C(CH₃)Ph), 0.86-1.58 (m, 10H, cyclohexyl —CH₂).

E-1-(4′-Cyclohexyl-3′-methyl-4′-oxo-3′-phenylbut-1-enyl)-4-(2-methoxyphenyl) piperazine,

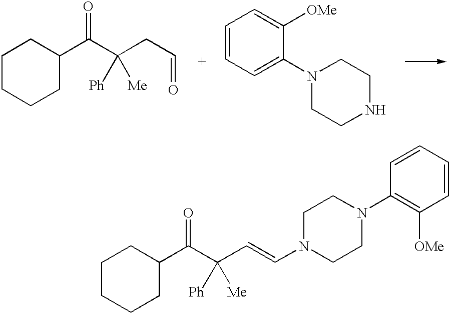

A solution of 0.98 g (0.0038 mol) of 4-cyclohexyl-3-methyl-4-oxo-3-phenyl-1-butanaldehyde in 1.8 mL of iPrOAc was added to 0.73 g (0.0038 mol) of neat 1-(2-methoxyphenylpiperizine. The mixture was stirred overnight at RT. Solid precipitate that had formed was vacuum filtered and washed twice with 2.5 mL of iPrOAc and air dried to afford 0.30 g (18 % %) of the tile compound as a yellow solid. The filtrate was concentrated to afford 1.38 g of the crude product. ¹H NMR (d⁶-DMSO): δ7.32-7.35 (m, 2H, phenyl CH), 7.22-7.25 (m, 3H, phenyl CH), 6.85-7.00 (m, 4H, phenyl CH), 6.04 (d, J=14.2 Hz, 1H, CR₃CH═CH NR₂(trans)), 4.95 (d, J=14.2 Hz, 1H, CR₃CH═CH NR₂(trans)), 3.77 (s, 3H, OCH₃), 2.98-3.21 (m, 8H, piperazine CH₂), 2.38-2.49 (m, 1H, cyclohexyl CH), 1.58-1.63 (m, 2H, cyclohexyl CH₂), 1.47-1.59 (m, 2H, cyclohexyl —CH₂), 1.36 (s, 3H, R₂C(CH₃)Ph), 1.21-1.34 (m, 3H, cyclohexyl —CH₂), 1.03-1.21 (m, 2H, cyclohexyl —CH₂), 0.83-1.03 (m, 1H, cyclohexyl —CH₂).

1-(4′-Cyclohexyl-3′-methyl-4′-oxo-3′-phenylbutyl)-4-(2-methoxyphenyl) piperazine

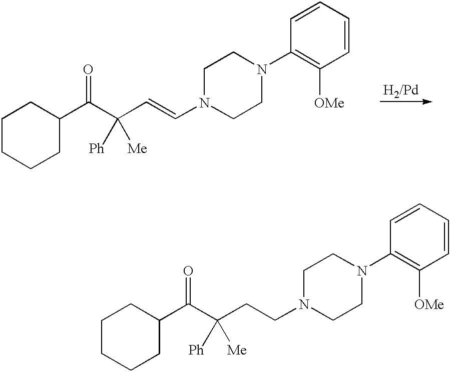

H₂ was introduced at 50 psi to a slurry of 0.049 g (0.023 mmol) of 5% Pd/C and 0.20 g (0.46 mmol) of E-1-(4′-Cyclohexyl-3′-methyl-4′-oxo-3′-phenylbut-1-enyl)-4-(2-methoxyphenyl) piperazine, in 10 mL of IPA and mixture was shaken overnight at RT to complete reaction. The black slurry was vacuum filtered and concentrated to afford 0.19 g of crude product residue. The residue was diluted with 20 mL of CH₂Cl₂, washed twice with 20 mL of 1N HCl (aq), washed with 20 mL of 1N NaOH (aq), washed with 20 mL of 25% NaCl (aq), dried over MgSO4, gravity filtered and concentrated to afford 0.15 g (75%) of product. ¹H NMR (d⁶-DMSO): δ7.35-7.37 (m, 2H, phenyl CH), 7.25-7.28 (m, 3H, phenyl CH), 6.87-6.92 (m, 2H, phenyl CH), 6.82-6.83 (m, 2H, phenyl CH), 3.72 (s, 3H, OCH₃), 2.80-2.95 (m, 4H, piperazine CH₂), 2.30-2.42 (m, 4H, piperazine CH₂), 1.94-2.13 (m, 4H), 1.50-1.55 (m, 3H, cyclohexyl CH₂), 1.48 (s, 3H, R₂C(CH₃)Ph), 1.36-1.40 (m, 1H, cyclohexyl —CH₂), 1.02-1.21 (m, 5H, cyclohexyl CH₂),0.84-0.98 (m, 1H, cyclohexyl CH₂).

### Table of Results

TABLE 1
Palladium-Catalyzed Coupling of α-Methyl-γ-butyrolactone
Enolate with Aryl bromides

| Aryl bromide | Product | Yield (%) | ee (%) | [α]_{D} (c, solvent) |
|---|---|---|---|---|
| 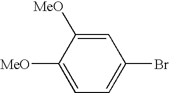 | 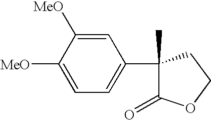 | 70 | 61 | −46.7 (1.02, CHCl₃) |
| 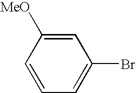 | 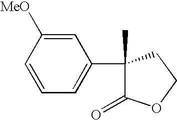 | 59 | 62 | −51.8 (1.00, MeOH)^{a} |
| 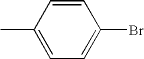 | 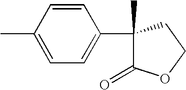 | 60 | 59 | −51.1 (1.03, CHCl₃)^{b} |
| 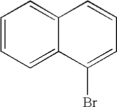 | 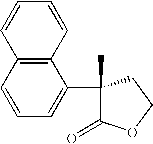 | 61 | 15 | −24.0 (1.03, CHCl₃) |
| 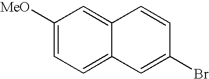 | 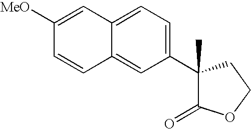 | 65 | 61 | −54.3 (1.03, CHCl₃) |
| 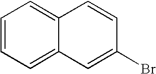 | 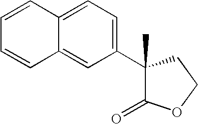 | 58 | 63 | −59.0 (0.90, CHCl₃) |
| 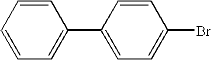 | 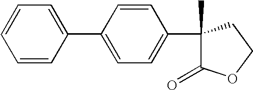 | 90 | 65 | −52.8 (1.06, CHCl₃) |
| 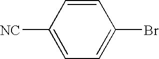 | 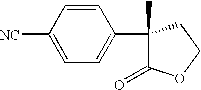 | 55 | 54 | −37.0 (1.53, CHCl₃) |

Yields reported are isolated ones. All compounds were characterized by NMR (¹H, ¹³C). Enantiomeric excess was determined either by chiral HPLC or by ¹H-NMR with shift reagent Europium tris[3-(heptafluoropropylhydroxymethylene)-(+) -camphorate]. Reactions were carried out using 5-10 mol % of Pd(OAc)₂, 6.25-12.5 mol % of R-(+)-BINAP, 2 equiv. of aryl bromide, 1.5-1.75 equiv. of KN(TMS)2 and 1 equiv. of butyrolactone in toluene at 100-105° C. for 20-24 hours. a) The same compound prepared via a 7 steps procedure by starting from (S)-(−)-benzyl 2,3-epoxypropyl ether was reported in literature. [α]_{D}=−72.1 was reported for the enantiomeric pure compound⁴. b) The reaction was carried out at 40° C.

We claim:

1. A process for preparing a compound of the formula (I): 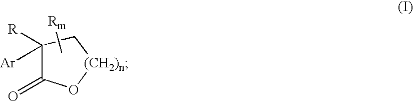
or a pharmaceutically acceptable salt thereof, wherein R is a H, alkyl, alkenyl, alkynyl, aryl, alkylaryl, arylalkyl, heteroalkyl, heteroaryl, heterocyclicaryl, carboxyalkyl, carboxyaryl, cyano, carbocyclic, or heterocyclic radical; Ar is an aryl group;
Rₘ is a single or multiple substituent on the lactone ring other than at the α-position; and n is 1-20, comprising the steps of:
(a) stirring a mixture of a palladium source and a ligand in a suitable solvent;
(b) adding a compound of formula ArX, wherein Ar is aryl, X is the anion of a strong acid or a leaving group selected from the group comprising Br, Cl, I, OSO₂ CₙF₂ₙ₊₁, and OP(O)(OCH₂ₙ₊₁)₂;
(c) adding a lactone;
(d) adding a suitable base
(e) isolating the compound of formula I;
(e) optionally preparing a pharmaceutically acceptable acid salt of the compound of formula I.

2. A process according to claim 1 wherein ArX is an arylbromide.

3. A process according to claim 1 wherein the ligand is a chiral ligand.

4. A process according to claim 1 wherein R is selected from the group consisting of C₁-C₈ alkyl, aryl and heterocycle.

5. A process according to claim 1 wherein the solvent is toluene or tetrahydrofuran.

6. A process according to claim 1 wherein Rm is a single substituent.

7. A process according to claim 1 wherein n is 1 or 2, or 3.

8. A process according to claim 1 wherein the mixture is heated at a temperature from about 40-110° C. for about 16-30 hours.

9. A process according to claim 1 wherein the palladium source is palladium acetate.

10. A process according to claim 1 wherein a suitable base is an organic base.

11. A process according to claim 9 wherein the organic base is selected from the group consisting of potassium bis(trimethylsilyl)amide, potassium amide, lithium diisoprpopylamide.

12. A process for the preparation of a compound of formula (II) 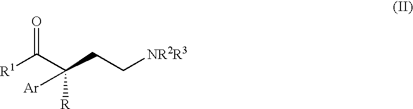
wherein R and R¹ are hydrogen, alkyl, alkenyl, alkynyl, aryl, alkylaryl, arylalkyl, heteroalkyl, heteroaryl, carboxyalkyl, carboxyaryl, cyano, carbocyclic or heterocyclic radical or combine to form a substituted or unsubstituted carbocycle or heterocycle; R² and R³ are independently selected from the group consisting of H, alkyl, alkenyl, alkynyl, aryl, alkylaryl, arylalkyl, heteroalkyl, heteroaryl, carboxyalkyl, carboxyaryl, cyano, carbocyclic or heterocyclic radical or combine to form a substituted or unsubstituted carbocycle or heterocycle, comprising the steps of:
(a) arylating a compound of formula (a) 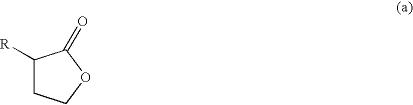
with an aryl halide (ArX) in the presence of a palladium source and a chiral ligand, in a suitable solvent to form a compound of formula (b) 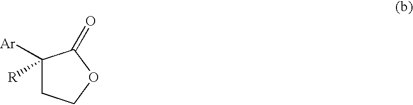
wherein R and Ar are as described;
(b) performing a 1,2-addition on the lactone to form the compound of formula (c), 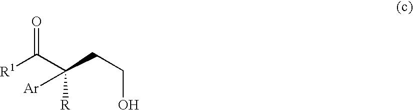
wherein R¹ is as described;
(c) oxidizing the compound (c) to afford the compound of formula (d) 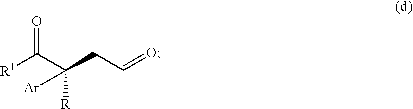
(d) performing a reductive amination using an amine (NHR²R³) and a reducing agent to form a compound of formula (II) 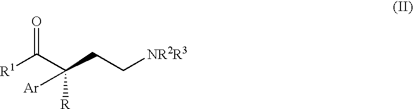
wherein R, R¹, R² and R³ are as described; and
(e) optionally forming a salt of a compound of formula (II).

13. A process according to claim 11 wherein the palladium source is palladium acetate.

14. A process according to claim 11 wherein the chiral ligand is R or S BINAP.

15. A process according to claim 11 wherein the aryl halide is a substituted or unsubstituted phenyl bromide or naphthyl halide.

16. A process according to claim 11 wherein the aryl halide (ArX) is phenyl bromide.

17. A process according to claim 11 wherein the 1,2- addition is a Grignard reaction using the reagent R¹MgX.

18. A process according to claim 11 wherein R¹ is selected from the group consisting of an alkyl, phenyl, naphthyl, or thienyl group.

19. A process according to claim 11 wherein R¹ is the group cyclohexyl.

20. A process according to claim 11 for preparing a compound of formula (X′) 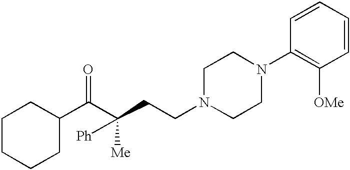
(X′)
wherein Ar is represented by phenyl, R is represented by methyl, R¹ is represented by cyclohexyl, and R² and R³ combine with the nitrogen to which they are attached to form the 4-(2-methoxyphenyl)piperazinyl group of formula (X′).
